(19) United States
(12) Patent Application Publication
(10) Pub. No.: US 2022/0218951 A1
(43) Pub. Date: Jul. 14, 2022

(54) CATHETER SECUREMENT COMPOSITIONS AND METHODS PROVIDING SIGNIFICANTLY STRONG SECUREMENT, WATER RESISTANT SEAL, EFFECTIVE HEMOSTATIS, AND ANTIBACTERIAL PROPERTIES AT AND AROUND VASCULAR ACCESS SITES

(71) Applicant: Adhezion Biomedical, LLC., Wyomissing, PA (US)

(72) Inventors: Sheng Zhang, Hickory, NC (US); Amanda Guido, Apex, NC (US)

(21) Appl. No.: 17/569,168

(22) Filed: Jan. 5, 2022

Related U.S. Application Data
(60) Provisional application No. 63/134,888, filed on Jan. 7, 2021.

(51) Int. Cl. A61M 25/02

(52) U.S. Cl. CPC A61M 25/02; A61M 2025/0273; A61M 2025/0253; A61M 2025/0246

(57) ABSTRACT

Catheter securement compositions and methods providing significantly strong securement, water resistant seal, effective hemostasis, and antibacterial properties at and around vascular access sites. By securing the insertion site and the hub to the skin, the composition and method reduce complications such as catheter dislodgement, catheter infiltration, catheter migration, catheter occlusion, catheter-related phlebitis, and catheter-related infections.

### RELATED APPLICATIONS

This application claims the benefit of priority to U.S. Provisional Patent Application Ser. No. 63/134,888, filed on Jan. 7, 2021, the contents of which are incorporated in this application by reference.

### BACKGROUND

In the US alone roughly 330 million intravascular devices are purchased by hospitals each year. 60% to 90% of hospitalized patients require an IV catheter during their hospital stay. Among these devices, peripheral venous catheters are the most frequently used, with an estimated 450 million catheter days annually. Because of the complications associated with IV catheter use, the development of reliable yet cost-effective securement techniques is extremely important.

One of the most common complications associated with catheter insertion includes migration, dislodgement, micromovement, and infiltration. Patient movement and frequent dressing changes can cause large-scale movement of the catheter, sometimes resulting in dislodgement and requiring a new catheter to be inserted. Small-scale catheter movement, also known as “pistoning,” irritates the vessel wall and is believed to lead to the development of phlebitis, the most common complication associated with catheter use. When secured by traditional catheter securement techniques, catheter failure rate is as high as 50%. Reducing the frequency of complications associated with catheter movement can, in turn, reduce the occurrence of needle stick injuries for health care workers and prevent otherwise unnecessary costs for hospital stays.

Another complication related to catheter insertion is catheter-related bloodstream infections (CRBSI). Common skin microbes, including coagulase-negative staphylococci, S. aureus, and enterococci, can migrate to the catheter insertion site and colonize the catheter hub and tubing. A study conducted on 1,681 IV catheters found that after their use 33.8% were colonized and 12.3% were infected, with roughly a third of the infected catheters corresponding to a bloodstream infection. Hospital-acquired infections (HAI) not only increase the length of a hospital stay but also the total cost; estimates put the extra cost of a hospital stay due to an HAI anywhere from $4,000 to $56,000 with 250,000-500,000 BSIs occurring annually in the US.

The ability to assist with hemostasis and the prevention of hematomas is one of the main criteria when selecting a catheter securement device. Complications due to bleeding at the insertion site for all types of intravascular catheters can cause frequent dressing changes, a need for gauze compressions at the site of insertion, and more serious complications, such as the development of hematomas that may lead to skin necrosis. Bleeding at the catheter insertion site is particularly critical for patients that are at a higher risk for bleeding. The severity of bleeding from the insertion site varies at different insertion site locations. As an example, bleeding complications for central venous catheters (CVC) are reported in up to 1.6% of cases that frequently require medical intervention. The current standard of care if bleeding occurs at the insertion site is to utilize gauze compressions and frequent dressing changes until the bleeding subsides. The ability to provide hemostasis at the insertion site has the potential to increase patient comfort while decreasing the overall length and cost associated with hospital stays. Therefore, it would be desirable to find a solution that can not only secure the intravascular catheter tubing and hub but also provide a hemostatic effect at the insertion site.

Development of catheter securement device that is able to prevent both macro- and micro-movements of the catheter components, stop bleeding of the catheter insertion site, as well as provide antibacterial property to reduce the risk of complications would be substantial. Conventional catheter dressing or accessory products can either provide securement ability, stop bleeding, or provide antibacterial property. As far as is known, there is no commercial product available that has all three properties prior to the development of the composition and device disclosed in the present invention. It is thus the purpose of the present patent to disclose a novel cyanoacrylate-based catheter securement device and composition, which can provide (a) a water-resistant barrier and seal on the catheter insertion site; (b) significantly stronger securement strength than conventional transparent dressing products; (c) hemostatic property to stop bleeding at the catheter insertion site; and (d) antibacterial property.

### SUMMARY

This invention provides compositions and methods which can significantly increase the securement strength of catheters, the peel strength of transparent dressing products over catheters, provide antibacterial property and thus potentially reduce blood stream infections, are capable of providing effective hemostasis and sealing at the catheter insertion site, and have a shelf life stability of at least 24 months post gamma and ETO sterilization.

The adhesive compositions and methods disclosed in this invention can be applied to devices including, but are not limited to, connector fittings, catheter systems (e.g., including catheters, catheter hubs, catheter adaptors, catheter tubing, etc.), fluid supply lines, inserted ports, other similar articles, or combinations thereof.

Compared to conventional and commercially available catheter dressing products, the liquid adhesive disclosed in this invention provides a much stronger securement strength in terms of catheter securement. The catheter securement strength of liquid adhesive compositions disclosed in this invention is about 10 times stronger than some of the competitor products, indicating the significant advantage of liquid adhesive disclosed in this invention over conventional catheter dressing products. The significantly stronger securement strength of the liquid adhesive composition disclosed in this invention can inhibit and/or reduce catheter movement, migration, and dislodgement of the catheter, which is a significant benefit of the liquid adhesive composition disclosed in this invention over the conventional and commercially available catheter dressing products.

Applying the liquid adhesive composition disclosed in the present patent underneath the dressing products can further significantly increase the securement strength of catheters. Applying the liquid adhesive composition disclosed in this invention underneath the catheter insertion tube, catheter hub, catheter wings, and transparent dressing product can provide a securement strength of at least 11 lbf to the catheter being secured, which is up to 36 times stronger compared to the securement strength provided by some of conventional dressing or tape products used alone.

The securement strength of the liquid adhesive composition disclosed in the present patent can maintain over a period of time. An in vitro study demonstrated that the securement strength of the liquid adhesive composition disclosed in this invention can provide the effective securement for at least 7 days.

The liquid adhesive composition disclosed in this invention can secure the catheter insertion tube and seal the insertion site, while conventional catheter dressing products can only secure the catheter hub/wing but leave the insertion tube unsecured and the insertion site unsealed, which is the most vulnerable part of the entire vascular access system.

Liquid adhesive composition disclosed in this invention can provide an effective and water-resistant seal on the catheter insertion site. The effective and water-resistant seal on the catheter insertion site of the liquid adhesive composition disclosed in this invention was evaluated by a liquid dye penetration method. The liquid adhesive composition disclosed in this invention provides hemostasis and stops bleeding at the catheter insertion site. The hemostatic property of the liquid adhesive composition disclosed in the present patent was confirmed by both modified activated clotting time assay and blood flow inhibition assay methods.

The liquid adhesive composition disclosed in this invention can provide antibacterial and bacteria immobilization properties, which are beneficial to catheter securement in terms of potentially inhibiting or reducing the known complication of the catheter related blood stream infection (CRBSI).

The liquid adhesive compositions disclosed in this invention can seal vascular access sites and secure vascular access devices providing antibacterial properties and hemostatic properties at or around vascular access sites, which typically requires the use of three or more different conventional products to achieve.

Sterilization of the liquid adhesive composition disclosed herein can be accomplished by any method, including, but not limited to chemical, physical and irradiation techniques. Liquid adhesive composition disclosed in this invention maintain a shelf life of at least 24 months post sterilization as evaluated by real time shelf life stability study.

The method described herein provides for securing a vascular access device which has a tube that has been inserted into a vascular system of a patient at a vascular access point. The method includes applying a first amount of a radiation-sterilized cyanoacrylate adhesive composition over the vascular access point. This first amount is allowed to cure to create a first cured radiation-sterilized cyanoacrylate adhesive composition. This first cured radiation-sterilized cyanoacrylate adhesive composition is transparent and provides a water-resistant seal barrier, a hemostatic effect on the vascular access point, an antimicrobial function to immobilize and eliminate bacteria at and around vascular access point. It also secures the insertion tube to the vascular access site. Next, a second amount of the radiation-sterilized cyanoacrylate adhesive composition is applied under a portion of the vascular access device at a site remote from the vascular access point. This second amount is allowed to cure thereby securing the vascular access device to the patient with a secured strength greater than 2 pounds of force (lbf).

In certain embodiments, the water-resistant seal barrier lasts for more than 3 days.

In certain embodiments, the vascular access device is a catheter and the first cured radiation-sterilized cyanoacrylate adhesive composition further inhibits complications associated with catheter insertion selected from the group consisting of catheter dislodgement, catheter infiltration, catheter migration, catheterocclusion, catheter-related phlebitis, and catheter-related infections.

In certain embodiments, the first cured radiation-sterilized cyanoacrylate adhesive composition further prevents bleeding at the vascular access point.

In certain embodiments, the first cured radiation-sterilized cyanoacrylate adhesive composition immobilizes bacteria at the vascular access point.

In certain embodiments, the first cured radiation-sterilized cyanoacrylate adhesive composition is an antimicrobial that provides at least a 6 log reduction of gram-positive bacteria, gram-negative bacteria, yeast, and fungi 3 minutes after curing.

In certain embodiments, the portion of the vascular access device under which second amount of the radiation-sterilized cyanoacrylate adhesive composition are wings.

In certain embodiments, the secured strength of the secured vascular access device to the patient is greater than 3 lbfs or 5 lbfs.

In certain embodiments, the vascular access device is selected from a group consisting of: Tegaderm dressing products, Opsite, Sorbaview Shield, Sorbaview Ultimate Window Dressing product line, SureView IV Securement Transparent Film Dressing, Venti-Gard, UltraDrape—UGPIV Barrier and Securement, and Securis Stabilization Devices.

In certain embodiments, the radiation-sterilized cyanoacrylate adhesive composition comprises a mixture of stabilized and sterile polymerizable monomers. In such embodiments, the polymerizable monomers comprise a cyanoacrylate with straight chain or branched chain alkyl or alkoxyalkyl groups having 4 to 20 carbon atoms, including but not limited to, 2-octyl cyanoacrylate, dodecyl cyanoacrylate, undecyl cyanoacrylate, decyl cyanoacrylate, butyl cyanoacrylate, nonyl cyanoacrylate, heptyl cyanoacrylate, hexyl cyanoacrylate, pentyl cyanoacrylate, propyl cyanoacrylate, ethyl cyanoacrylate, 2-ethylhexyl cyanoacrylate, methyl cyanoacrylate, 3-methoxybutyl cyanoacrylate, 2-butoxyethyl cyanoacrylate, 2-isopropoxyethyl cyanoacrylate, or 1-methoxy-2-propyl cyanoacrylate, or a combination thereof. For example, they may be butyl cyanoacrylate, octyl cyanoacrylate, or a combination thereof. Furthermore, the radiation-sterilized cyanoacrylate adhesive composition may be stabilized by free radical stabilizers and anionic stabilizers. In addition, the radiation-sterilized cyanoacrylate adhesive composition may be sterilized by irradiation methods and/or chemical sterilization methods. Finally, the radiation-sterilized cyanoacrylate adhesive composition may have a shelf life of at least 24 months post sterilization evaluated by real time shelf stability studies.

In certain embodiments, the vascular access device is selected from a group consisting of peripheral IV catheters, PICC catheters, and CVC catheters.

In certain embodiments, the radiation-sterilized cyanoacrylate adhesive provides securement of the vascular access device to the patient for up to 7 days.

In certain embodiments, the radiation-sterilized cyanoacrylate adhesive is packaged in an applicator. In such embodiments, the applicator may be fabricated from a material that is substantially impermeable to moisture and air.

### DESCRIPTION

The adhesive composition disclosed in the present patent can be accurately dispensed and applied to the catheter insertion site, on and underneath the catheter tubing, and underneath the catheter hub, which conventional catheter dressing products cannot achieve. Liquid adhesive dispensed by the applicator provides an effective securement of catheters such as intravenous (IV) catheters; peripheral venous catheters (PVCs), central venous catheters (CVCs), peripherally inserted central catheters (PICCs), arterial catheters, urinary catheters, and dialysis catheters.

The adhesive composition disclosed in the present patent will be applied to devices including, but are not limited to, connector fittings, catheter systems (e.g., including catheters, catheter hubs, catheter adaptors, catheter tubing, etc.), fluid supply lines, inserted ports, other similar articles, or combinations thereof. Examples of catheter systems can include, but are not limited to, intravenous (IV) catheters; peripheral venous catheters (PVCs), central venous catheters (CVCs), peripherally inserted central catheters (PICCs), arterial catheters, urinary catheters, and dialysis catheters. Most common commercially available peripheral catheters include, but are not limited to, Insyte Autoguard from BD, Nexiva IV catheter from BD, BD Saf-T-Intima, Jelco Viavalve safety Iv Catheter, Excel Safelet Catheters, Retractable Technologies VanishPoint catheters, Smiths Medical Protectiv Safety IV catheter, McKesson Select catheter, Bard Access Accucath, Terumo Medical Surflo, and B Braun Introcan Safety IV Catheter. The most common commercially available midline peripheral catheters include, but are not limited to, Silicon First Midcath by Med Alliance Group, Argon Medical Midline Single Lumen, Bard Power Glide Midline, First Midcath, and Arrowg+ard Blue advance midline. The most common commercially available PICC include, but are not limited to, PowerPicc by BD, BioFlo PICC by Angiodynamics, Argyle PICC by Covidien, and Arrow International PICC TwinCath. In order to keep the catheter, tubing, or other medical line properly positioned for the duration of treatment, the catheter, tubing, or medical line can be secured to the patient with the adhesive compositions, which is simple to use while providing reliable fixation of the catheter to the skin of the patient.

Compared to conventional and commercially available catheter dressing products, which include, but are not limited to, the Tegaderm™, Opsite, HubGuard®, and Durapore products, the liquid adhesives disclosed in the present patent provide a much stronger securement strength in terms of catheter securement. As shown in Table 1, the liquid adhesive compositions disclosed in the present patent provided much stronger securement strength in terms of securing BD Nexiva catheter when compared to conventional transparent dressing or tape products. Table 1 indicates the securement strength is about 10 times stronger than some of the competitor products indicating the significant advantage of liquid adhesive disclosed in the present patent over conventional catheter dressing products. Specifically, Table 1 shows the securement strength of the liquid adhesive compositions disclosed in the present patent versus conventional catheter securement dressing products after 30 minutes while securing Nexiva catheters.

TABLE 1

| | Securement |
|---|---|
| Securement Method | Strength (1bf.) |
| Adhesive composition | 3.008 |
| disclosed in the present patent | |
| Tegaderm 1683 | 1.594 |
| Opsite | 0.313 |
| Hubguard | 0.349 |
| Durapore | 0.303 |

The significantly stronger securement strength of the liquid adhesive composition disclosed in the present patent can inhibit and/or reduce catheter movement, migration, and dislodgement of the catheter, which is a significant benefit of the liquid adhesive composition disclosed in the present patent over the conventional and commercially available catheter dress products, which include, but not limited to, Tegaderm™, Opsite, HubGuard®, Durapore, 3M PICC/CVC Securement, SorbaviewShield, Sorbaview Ultimate Window Dressing product line, SureView IV Securement TransparentFilm Dressing, Venti-Gard, UltraDrape—UGPIV Barrier and Securement, Securis Stabilization Device. Tegaderm is the most popular film dressing product line. Commercially available Tegaderm products are Tegaderm CHG 1659 IV Securement Dressing, Tegaderm CHG 1658 IV Securement Dressing, Tegaderm 1683 IV Advanced Securement Dressing, Tegaderm 1685 IV Advanced Securement Dressing, Tegaderm 1688 IV Advanced Securement Dressing, Tegaderm 1689 IV Advanced Securement Dressing, Tegaderm 1680 IV Advanced Securement Dressing for Pediatrics, Tegaderm 1682 IV Advanced Securement for Pediatrics, Tegaderm Roll Film dressing, and Tegaderm HP 9519HP. Conventional catheter stabilization devices that are commercially available are, GRIP-LOK by Baxter, WingGuard Catheter Securement Device, Grip-Lok by TidiProducts, and the StatLock product line.

Another advantage of the liquid adhesive composition disclosed in the present patent over conventional and commercially available catheter dressing products is that the liquid adhesive composition disclosed in the present patent can secure the catheter insertion tube and seal the insertion site, while conventional catheter dressing products can only secure the catheter hub/wing but leave the insertion tube unsecured and the insertion site unsealed, which is the most vulnerable part of the entire vascular access system. Unsecured insertion tube and unsealed catheter insertion site could be the most obvious reason that would cause migration, dislodgement, micromovement, phlebitis, and catheter-related blood stream infections (CRBSI).

The securement strength of the liquid adhesive composition disclosed in the present patent can maintain over a period of time. In one embodiment, the liquid adhesive composition disclosed in the present patent can provide the effective securement for at least 1 day. In one preferred embodiment, the securement strength of the liquid adhesive composition disclosed in the present patent can provide the effective securement for at least 3 days. In another preferred embodiment, the securement strength of the liquid adhesive composition disclosed in the present patent can provide the effective securement for at least 5 days, and in one more preferred embodiment, the securement strength of the liquid adhesive composition disclosed in the present patent can provide the effective securement for at least 7 days. As an example, the securement strength of the liquid adhesive composition disclosed in the present patent evaluated at time intervals of 1 day, 3 days, and 7 days was 3.06 lbf, 3.02 lbf, and 3.72 lbf, respectively.

The liquid adhesive composition disclosed in the present patent are also compatible with conventional catheter dressing products, which include, but are not limited to, Tegaderm™, Opsite, HubGuard®, Durapore, 3M PICC/CVC Securement, Sorbaview Shield, Sorbaview Ultimate Window Dressing product line, SureView IV Securement Transparent Film Dressing, Venti-Gard, UltraDrape—UGPIV Barrier and Securement, Securis Stabilization Device.

The combination use of the liquid adhesive composition disclosed in the present patent and conventional catheter dressing products are feasible, which are proved to be significantly more effective than conventional catheters dressing products used alone. As demonstrated in Table 2, the securement strengths of the liquid adhesive composition disclosed in the present patent plus different dressing products are significantly greater than those provided by conventional catheter dressing products used alone. Specifically, Table 2 shows the securement strength of the liquid adhesive composition disclosed in the present patents. adhesive composition and conventional products for securing a Nexiva I.V. Catheters for 30 minutes.

TABLE 2

| | Securement |
|---|---|
| Securement Method | Strength (1bf.) |
| Tegaderm I.V. 9525 | 1.59 |
| Opsite | 0.31 |
| Hub guard | 0.35 |
| Durapore | 0.30 |
| Adhesive composition disclosed | 3.35 |
| herein + Tegaderm I.V. 9525 | |
| Adhesive composition disclosed herein + Opsite | 3.60 |
| Adhesive composition disclosed herein + Hubguard | 2.44 |
| Adhesive composition disclosed herein + Durapore | 3.38 |

The effective securement strength of the liquid adhesive composition disclosed in the present patent was also confirmed by an in vivo model. A total of six canines were divided into 3 groups with 2 animal per group to evaluate the stress of Autoguard IV catheters, Power PICC catheters, or Arrowguard CVC catheters secured by the liquid adhesive composition disclosed in the present patent. The dynamic challenge was to pull on the cannula 10 times each hour for 6 hours. The study showed no securement failure for each of the cannula tested at any time interval, which demonstrated that the liquid adhesive composition disclosed in the present patent successfully secured all three types of catheters tested in peripheral and central veins.

Another advantage is that the liquid adhesive composition disclosed in the present patent can also significantly increase the peel strength of transparent dressing products, which include, but are not limited to, Tegaderm dressing products, Opsite, Sorbaview Shield, Sorbaview Ultimate Window Dressing product line, SureView IV Securement Transparent Film Dressing, Venti-Gard, 3M PICC/CVC Securement, UltraDrape—UGPIV Barrier and Securement, and Securis Stabilization Device. Tegaderm dressing products include, but are not limited to, Tegaderm CHG 1659 IV Securement Dressing, Tegaderm CHG 1658 IV Securement Dressing, Tegaderm 1683 IV Advanced Securement Dressing, Tegaderm 1685 IV Advanced Securement Dressing, Tegaderm 1688 IV Advanced Securement Dressing, Tegaderm 1689 IV Advanced Securement Dressing, Tegaderm 1680 IV Advanced Securement Dressing for Pediatrics, Tegaderm 1682 IV Advanced Securement for Pediatrics, Tegaderm Roll Film dressing, and Tegaderm HP 9519HP. The liquid adhesive composition disclosed in the present patent can be applied partially or entirely underneath the dressing products. In one embodiment, the peel strength of the transparent dressing products with the liquid adhesive composition disclosed in the present patent applied underneath is about 2 times stronger than that of the transparent dressing product alone. In another embodiment, the peel strength of the transparent dressing products with the liquid adhesive composition disclosed in the present patent applied underneath is about 4 times stronger than that of the transparent dressing product alone. In a preferred embodiment, the peel strength of the transparent dressing products with the liquid adhesive composition disclosed in the present patent applied underneath is about 8 times stronger than that of the transparent dressing product alone. In a more preferred embodiment, the peel strength of the transparent dressing products with the liquid adhesive composition disclosed in the present patent applied underneath is about 8 times stronger than that of the transparent dressing product alone.

Applying the liquid adhesive composition disclosed in the present patent underneath the dressingproducts can also significantly increase the securement strength of catheters that include, but are not limited to peripheral intravenous catheters (PICs), central venous catheters (CVCs), peripherally inserted central catheters (PICCs), arterial catheters, urinary catheters, and dialysis catheters, as demonstrated by Table 3 below. In one embodiment, applying the liquid adhesive composition disclosed in the present patent underneath the catheter insertion tube, catheter hub, catheter wings, and transparent dressing product can provide a securement strength of at least 5 lbf to the catheter being secured. In another embodiment, applying the liquid adhesive composition disclosed in the present patent underneath the catheter insertion tube, catheter hub, catheter wings, and transparent dressing product can provide a securement strength of at least 6 lbf to the catheter being secured. In another embodiment, applying the liquid adhesive composition disclosed in the present patent underneath the catheter insertion tube, catheter hub, catheter wings, and transparent dressing product can provide a securement strength of at least 8 lbf to the catheter being secured.

In a preferred embodiment, applying the liquid adhesive composition disclosed in the present patent underneath the catheter insertion tube, catheter hub, catheter wings, and transparent dressing product can provide a securement strength of at least 10 lbf to the catheter being secured.

In a preferred embodiment, applying the liquid adhesive composition disclosed in the present patent underneath the catheter insertion tube, catheter hub, catheter wings, and transparent dressing product can provide a securement strength of at least 11 lbf to the catheter being secured. Specifically, Table 3 shows securement strength of transparent dressing product 5 alone, and transparent dressing product 5 plus adhesive composition disclosed in the present patent with additional drops of adhesive composition applied directly under transparent dressing product 5 while securing BD Nexiva catheters onto pig skin for 30 minutes.

TABLE 3

| | Securement |
|---|---|
| Securement Method | Strength (1bf.) |
| Secured by Tegaderm I.V. 9525 alone | 1.6 |
| 4 drops of adhesive composition disclosed herein | 10.8 |
| applied to the windowarea of Tegaderm I.V. 9525, | |
| immediately applying Tegaderm I.V. 9525 | |
| 8 drops of adhesive composition disclosed | 11.8 |
| herein applied to the window area of Tegaderm I.V. 9525, | |
| immediately applying Tegaderm I.V. 9525 | |
| Secured by adhesive composition disclosed herein alone | 3.0 |
| Secured by adhesive composition disclosed | 3.3 |
| herein + Tegaderm I.V. 9525, no additional adhesive | |
| applied to the window area of Tegaderm I.V. 9525 | |

Another advantage of the liquid adhesive composition disclosed in the present patent over conventional catheter dressing products is that the catheter securement device and composition disclosed herein can provide an effective and water-resistant seal on the catheter insertion site.

In one embodiment, the liquid adhesive composition disclosed in the present patent can provide the effective and water-resistant seal on the catheter insertion site for at least 1 day. In one preferred embodiment, the securement strength of the liquid adhesive composition disclosed in the present patent can provide the effective and water-resistant seal on the catheter insertion site for at least 3 days. In another preferred embodiment, the securement strength of the liquid adhesive composition disclosed in the present patent can provide the effective and water-resistant seal on the catheter insertion site for at least 5 days, and in one more preferred embodiment, the securement strength of the liquid adhesive composition disclosed in the present patent can provide the effective and water-resistant seal on the catheter insertion site at least 7 days.

The effective and water-resistant seal on the catheter insertion site of the liquid adhesive composition disclosed in the present patent was evaluated by a liquid dye penetration method. Once the catheters have been inserted into the skin, they are secured to the skin by applying the liquid adhesive composition disclosed in the present patent. Once the appropriate amount of time has passed, a total of thirty minutes, the aqueous liquid dye is applied on top of the point in which the catheter tubing is inserted into the skin. Once this is completed the liquid dye rested for certain time intervals, the aqueous liquid dye solution was completely removed from the testing articles secured by the liquid adhesive composition disclosed in the present patent by using a spray bottle filled with water. Observations were recorded after initial removal of the dye, underneath the hub, and on the insertion point. Observations were also recorded by looking underneath a magnifying glass to evaluate the sealant integrity after initial removal of the dye, underneath the hub, and on the insertion point. For the control article, the dye was not able to be removed using water, because the dye had integrated with the skin and was unable to be removed. If there is no film barrier of the liquid adhesive composition disclosed in the present patent, then the skin will absorb the dye. The test results demonstrated that the liquid adhesive composition disclosed in the present patent provides an effective barrier against aqueous solutions. The water based liquid dye did not penetrate the deployed film at the junction of the catheter and the skin. Therefore, the liquid adhesive composition disclosed in the present patent provides securement to the catheter while providing a proficient sealant layer for the insertion site as well as the catheter hub.

The adhesive composition of the present invention has shown significant resistance to water penetration tested by the hydrostatic pressure impact test. The adhesive composition of the present invention provides an effective barrier against aqueous solutions, and the sealant integrity of the adhesive film layer once applied to the catheter stays intact with no pinholes. The water-based liquid dye did not penetrate the adhesive film at the junction of the catheter and the skin; therefore, the adhesive composition of the present invention provides securement to the catheter while providing a proficient sealant layer for the insertion site as well as the catheter hub.

Compared to conventional and commercially available catheter dressing products, yet another advantage of the liquid adhesive composition disclosed in the present patent is that the liquid compositions disclosed herein provide hemostasis to the catheter insertion site. The hemostatic property of the liquid adhesive composition disclosed in the present patent was confirmed by both modified activated clotting time assay and blood flow inhibition assay methods.

An experiment was conducted to determine the hemostatic properties of the liquid adhesive composition disclosed in the present patent through means of customized modified activated clotting time (mACT). The liquid adhesive composition disclosed in the present patent was used to study the hemostatic properties while in contact with blood or plasma. Saline was the negative control used in all experiments. Each test group was evaluated when in contact with citrated whole blood (diluted 1:1 with saline), platelet poor plasma (prepared via centrifugation of citrated whole blood at 2500×g for 20 minutes at 21° c.), and non-anticoagulated whole blood (diluted 1:1 with saline) each from four healthy donors. A clotting analyzer was used on blood samples collected into sodium citrate anticoagulant or non-additive vacutainer tubes to evaluate the effects of the liquid adhesive composition disclosed in the present patent on mACT assay. A certain amount of diluted whole blood (WB) or neat platelet poor plasma (PPP) were added to each well. A control standard prothrombin time (PT) test with normal pooled plasma and Neoplastine CI plus regent was performed each day of testing for control. Inside the incubation wells, a test strip was placed for about 3 minutes before being transferred to the test wells. When the liquid adhesive composition disclosed in the present patent was deployed, the test was initiated with a maximum time limit of 180 seconds. To provide a comparison with a known hemostatic agent, thromboplastin, or tissue factor, WB was also placed into contact with Neoplastine CI plus reagent. This test was performed with results presented as the amount of time (seconds) it took to achieve hemostasis with the liquid adhesive composition disclosed in the present patent in the presence of WB or other blood products.

The modified activated clotting time analysis indicated that the liquid adhesive composition disclosed in the present patent is significantly faster to achieve hemostasis of whole blood (WB), neat platelet poor plasma (PPP) or other blood products, than other blood coagulation agents including but not limited to Thromboplastin. In one embodiment, the liquid adhesive composition disclosed in the present patent is at least 3 times faster to achieve hemostasis than other blood coagulation agents. In a preferred embodiment, the liquid adhesive composition disclosed in the present patent is at least 6 times faster to achieve hemostasis than other blood coagulation agents. In another preferred embodiment, the liquid adhesive composition disclosed in the present patent is at least 9 times faster to achieve hemostasis than other blood coagulation agents. In a more preferred embodiment, the liquid adhesive composition disclosed in the present patent is at least 12 times faster to achieve hemostasis than other blood coagulation agents.

The hemostatic properties of the liquid adhesive composition disclosed in the present patent was also evaluated by blood flow inhibition (BFI) assay. Three sodium citrate vacutainer tubes or one non-additive tube were used to collect blood. A peristaltic pump was installed with two pieces of four-inch C-flex tubing added to the distal and proximal ends of the peristaltic pump tube, which contains a 1.5 mL Eppendorf tube. One milliliter of citrated diluted whole blood (WB), non-anticoagulated WB, or Platelet-Poor Plasma (PPP) was perfused through the tubing until the blood reached the distal end. The liquid adhesive composition disclosed in the present patent was applied to the tubing tip, once the blood reached the distal end of the tubing. Once blood flow stopped, the pump was stopped, the tubing was removed, and the Eppendorf tube containing the collected blood was weighed. The average weights of citrate anticoagulated WB, non-anticoagulated WB, or Platelet-Poor Plasma (PPP) collection contents after coming in contact with the liquid adhesive composition disclosed in the present patent are significantly less than those after coming in contact with saline. The liquid adhesive composition disclosed in the present patent is statistically significant in halting excessive blood flow compared to the control or no treatment group.

An in vivo study was also conducted to show the hemostasis capability of the liquid adhesive composition disclosed in the present patent in direct comparison to known hemostatic agents including Gelfoam® Powder and Kaltostat®. Each agent was applied to dermal incisions in heparinized swine. Each incision was full-thickness and linear with a length between 7 mm-10 mm. Each incision was either treated with the liquid adhesive composition disclosed in the present patent, Gelfoam® Powder, Kaltostat®, or with the sham control, upon which a hemostasis score was observed. The swine were induced under general anesthesia; the hair located on the dorsal-lateral back was removed; and fluids were administered to maintain or increase systolic blood pressure, thereby achieving adequate blood flow. Heparin was administered to achieve adequate blood flow from each incision. The spot for each incision was outlined on the back of each swine using a marker and ruler. There were thirty sites located on each side of the back for each pig; the incisions were lined in three rows of ten. The treatments that each incision site received were rotated in a repeating order as follows: Sham Control, Gelfoam® Powder, Kaltostat®, and the liquid adhesive composition disclosed in the present patent. The rotation continued until all incisions were treated. The bleeding scores prior to the treatment serve as a baseline for start of bleeding to determine the effect the different articles have on improving hemostasis of the incisions. Prior to treatment with the liquid adhesive composition disclosed in the present patent, the average incision bleeding score was 2.3±1.0, while the bleeding score was significantly dropped to 0.2±0.4 after treating the incision with the liquid adhesive composition disclosed in the present patent. The statistical analysis preformed shows that the liquid adhesive composition disclosed in the present patent is statistically equivalent to Gelfoam® and Kaltostat® in terms of providing hemostatic effect on swine full thickness incisions.

Another advantage is that the liquid adhesive composition disclosed in the present patent can provide the antibacterial and bacteria immobilization property, which is beneficial to catheter securement in terms of potentially inhibiting or reducing the known complication of the catheter related blood stream infection (CRBSI). Studies were conducted to test the supposition that the liquid adhesive composition disclosed in the present patent immobilizes microorganisms thereby preventing the spread of microorganisms. Microorganisms chosen for examination individually in these experiments, include, but are not limited to, Methicillin Resistant Staphylococcus Aureus (MRSA), S. epidermidis, Pseudomonas aeruginosa, Candida albicans and Corynebaterium species. The experiments demonstrated that the liquid adhesive composition disclosed in the present patent was effective in preventing migration of microorganisms into an incision site. Mitigation of more than 3.9 log 10 for each of the five test microorganisms was observed.

Another study was conducted to appraise the bacterial immobilization properties of the liquid adhesive composition disclosed in the present patent on human volunteers. Four treatments —blank control, the liquid adhesive composition disclosed in the present patent, Betadine (positive control), and the liquid adhesive composition disclosed in the present patent plus Betadine, respectively—were applied on a randomized trial consisting of sixty (60) healthy male and female volunteers. The test results indicated the liquid adhesive composition disclosed in the present patent was effective in significantly reducing microbial colonization within 15 minutes of application (99.9%) and maintaining a low microbial colonization throughout the 24 hours post the treatment.

The liquid adhesive composition disclosed in the present patent was tested in its liquid state against the challenge microorganisms such as Escherichia coli, Klebsiella pheumoniae, Stphylococcus epidermidis, and Staphylococcus aureus subs. Aureus (MRSA). The liquid adhesive composition disclosed in the present patent was determined to be an effective antimicrobial agent. In one embodiment, the kill level observed for the liquid adhesive composition disclosed in the present patent after a three-minute contact time can be about five log reduction. In another embodiment, the kill level observed for the liquid adhesive composition disclosed in the present patent after a three-minute contact time can be about 6 log reduction. In another embodiment, the kill level observed for the liquid adhesive composition disclosed in the present patent after a three-minute contact time can be about 7 log reduction. In a preferred embodiment, the kill level observed for the liquid adhesive composition disclosed in the present patent after a three-minute contact time can be about 8 log reduction. In a more preferred embodiment, the kill level observed for the liquid adhesive composition disclosed in the present patent after a three-minute contact time can be about 9 log reduction.

The liquid adhesive compositions disclosed in this invention can seal vascular access sites and secure vascular access devices providing antibacterial property and stopping bleeding at or around vascular access sites, which requires three or more different conventional products to achieve. In other words, the liquid adhesive compositions disclosed in this invention possess several characteristics that are critical for the maintenance and care of the vascular access sites and the stabilization and securement of vascular access devices. The liquid adhesive compositions disclosed in this invention can provide a water-resistant seal and barrier to the vascular access site up to 7 days while ensuring the vascular access sites visible during entire time of securement. The conventional catheter dressing products could not seal the vascular access sites, but leave the vulnerable vascular access sites open instead. The liquid adhesive compositions disclosed in this invention provide significantly stronger securement strength to the catheters than that provided by the conventional securement methods using the dressing products or stabilization devices, which can inhibit, reduce, or prevent catheter-related complications such as catheter dislodgement, catheter infiltration, catheter migration/movement, catheter occlusion, catheter-related phlebitis, or catheter-related infections.

The adhesive composition disclosed in the present patent is used to apply liquid sealant to secure catheters on human skin. Preferred adhesive compositions are readily polymerizable, e.g., anionically polymerizable and/or free radical polymerizable. The adhesive compositions are preferably a 1,1-disubstituted ethylene monomer, e.g., a cyanoacrylate monomer. In a preferred embodiment, the adhesive compositions are based upon one or more polymerizable cyanoacrylate monomers, and/or reactive oligomers of cyanoacrylate. Such cyanoacrylate monomers are readily polymerizable, e.g., anionically polymerizable and/or free radical polymerizable, to form polymers. Cyanoacrylate monomers suitable for use in accordance with the present invention include, but are not limited to, 1,1-disubstituted ethylene monomers of the formula:

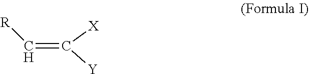

wherein X and Y are each strong electron withdrawing groups, and R is H, —CH═CH₂, or a C₁-C₄ alkyl group. Examples of monomers within the scope of Formula I include alpha-cyanoacrylates, vinylidene cyanides, C₁-C₄ alkyl homologues of vinylidene cyanides, dialkyl methylene malonates, acylacrylonitriles, vinyl sulfinates and vinyl sulfonates of the formula CH₂═CX′Y wherein X′ is —SO₂R′ or —SO₃R′ and Y′ is —CN, —COOR′, —COCH₃, —SO₂R′ or —SO₃R′, and R′ is H or hydrocarbyl. Preferred monomers of Formula I for use in this invention are alpha-cyanoacrylates. These monomers are known in the art and have the formula:

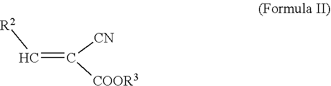

wherein R² is hydrogen and R³ is a hydrocarbyl or substituted hydrocarbyl group; a group having the formula —R⁴—O—R⁵—O—R⁶, wherein R⁴ is a 1,2-alkylene group having 2-4 carbon atoms, R⁵ is an alkylene group having 2-12 carbon atoms, and R⁶ is an alkyl group having 1-6 carbon atoms; or a group having the formula:

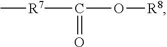

wherein R⁷ is:

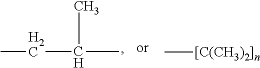

wherein n is 1-14, preferably 1-8 carbon atoms and R⁸ is an organic moiety.

Examples of suitable hydrocarbyl and substituted hydrocarbyl groups include straight chain or branched chain alkyl groups having 1-16 carbon atoms; straight chain or branched chain C₁-C₁₆ alkyl groups substituted with an acyloxy group, a haloalkyl group, an alkoxy group, a halogen atom, a cyano group, or a haloalkyl group; straight chain or branched chain alkenyl groups having 2 to 16 carbon atoms; straight chain or branched chain alkynyl groups having 2 to 12 carbon atoms cycloalkyl groups; arylalkyl groups; alkylaryl groups; and aryl groups. Table 4 below lists a few examples of securement strength of different cyanoacrylate compositions used for securing BD Autoguard catheters.

The organic moiety R⁸ may be substituted or unsubstituted and may be a straight chain, branched or cyclic, saturated, unsaturated, or aromatic. Examples of such organic moieties include C₁-C₈ alkyl moieties, C₂-C₈ alkenyl moieties, C₂-C₈ alkynyl moieties, C₃-C₁₂ cycloaliphatic moieties, aryl moieties such as phenyl and substituted phenyl, and arylalkyl moieties such as benzyl, methylbenzyl, and phenylethyl. Other organic moieties include substituted hydrocarbon moieties, such as halo (e.g., chloro-, fluoro-, and bromo-substituted hydrocarbons) and oxy- (e.g., alkoxy substituted hydrocarbons) substituted hydrocarbon moieties. Preferred organic radicals are alkylalkenyl and alkynyl moieties having from 1 to about 8 carbon atoms, and halo-substituted derivatives thereof. Particularly preferred are alkyl moieties of 4 to 8 carbon atoms. In the cyanoacrylate monomer of Formula II, R⁸ is preferably an alkyl group having 1-10 carbon atoms or a group having the formula -AO R⁹, wherein A is a divalent straight or branched chain alkylene or oxyalkylene moiety having 2-8 carbon atoms, and R⁹ is a straight or branched alkyl moiety having 1-8 carbon atoms. The preferred alpha-cyanoacrylate monomers used in this invention are 2-octyl cyanoacrylate, dodecyl cyanoacrylate, 2-ethylhexyl cyanoacrylate, butyl cyanoacrylate, methyl cyanoacrylate, 3-methoxybutyl cyanoacrylate, 2-butoxyethyl cyanoacrylate, 2-isopropoxyethyl cyanoacrylate, or 1-methoxy-2-propyl cyanoacrylate, or a combination thereof.

TABLE 4
Securement strength of different liquid adhesive
compositions disclosed in the present patent after
6 hours while securing BD Autoguard catheter.

| | Securement |
|---|---|
| Securement Method | Strength (1bf.) |
| Adhesive composition based upon | 2.69 |
| butyl cyanoacrylate (BCA) | |
| Adhesive composition containing less than 90% | 2.18 |
| octyl cyanoacrylate (OCA) | |
| Adhesive composition based upon | 3.30 |
| the mixture of OCA and BCA | |
| Adhesive composition based upon OCA | 3.95 |

In preferred embodiments of the present invention, the cyanoacrylate monomers can be prepared according to methods known in the art. Reference is made, for example, to U.S. Pat. Nos. 2,721,858 and 3,254,111, each of which is hereby incorporated in its entirety by reference. One such process includes, for example, reacting a cyanoacetate with formaldehyde in the presence of a basic condensation catalyst at an elevated temperature to produce a low molecular weight polymer. A de-polymerization (or cracking) step is followed under high temperature and high vacuum in the presence of acidic and anionic inhibitors, yielding a crude monomer that can be distilled under high temperature and high vacuum in the presence of radical and acidic inhibitors.

The distilled cyanoacrylate monomers can be filtered through one or multiple filters in order to reduce the bioburden level of the cyanoacrylate composition and remove any immiscible impurities or contaminants. If filtered, the cyanoacrylate monomers may be filtered through any suitable sized filters known in the art. For example, in a multiple step filtration process, the cyanoacrylate monomers may be filtered through a primary filter and one or more additional or secondary filters. The size of the primary filter may range, for example, on the order of about 0.01 to about 0.8 μm, preferably in the range of about 0.01 to about 0.6 μm, and more preferably in the range of about 0.03 to about 0.6 μm. The size of the additional or secondary filters may range, for example, on the order of about 1 to about 200 μm, preferably in the range of about 1 to about 150 μm, and more preferably in the range of about 1 to about 100 μm.

According to the embodiments of the present invention, cyanoacrylate monomer was produced with a high purity. The purity of cyanoacrylate in this invention is at least about 97% by weight, preferably at least about 98% by weight, and more preferably at least about 99% by weight. The purity of cyanoacrylate monomer was measured during and/or after distillation process. The high purity of cyanoacrylate monomer was obtained by multiple distillations under high vacuum and high temperature. The vacuum for distilling cyanoacrylate monomer is in the range of about 0.02 Torr to about 15 Torr, preferably in the range of about 0.05 Torr to about 10 Torr, and more preferably in the range of about 0.1 Torr to about 10 Torr. The distillation temperature is in the range of about 100° C. to about 180° C., preferably in the range of about 100° C. to about 160° C., and more preferably in the range of about 100° C. to about 150° C.

As will be recognized in the art, basic polymers or copolymers may be applied to reduce the amount of contaminants and extraneous additives in the cyanoacrylate monomer, but this can lead to several problems including premature polymerization. Some basic polymers or copolymers are not soluble in cyanoacrylate but are mixed with the monomer adhesive in mutual contact until the adhesive is destabilized. In order to achieve the mutual contact, such polymers or copolymers are mixed with the cyanoacrylate monomer for a minimum of 3 hours, which may remove possible acid residues to destabilize the adhesive. The solid powder of such polymer is then removed from cyanoacrylate adhesive by filtering.

According to the preferred embodiments of the present invention, a proper amount of both free radical and anionic stabilizers has to be included into said adhesive compositions in order to ensure that the inventive compositions do not cure upon sterilization, and further ensure that the inventive compositions can provide a stable shelf life of at least 24 months. The inventive compositions should be stabilized via the combination of free radical and anionic stabilizers. Other free radical stabilizers include without limitation, hydroquinone; catechol; hydroquinone monomethyl ether and hindered phenols such as butylated hydroxyanisol; 4-ethoxyphenol; butylated hydroxytoluene (BHT, 2,6-di-tert-butyl butylphenol), 4-methoxyphenol (MP); 3-methoxyphenol; 2-tertbutyl-4methoxyphenol; 2-tert-butyl-4-methoxyphenol; 2,2-methylene-bis-(4-methyl-6-tert-butylphenol). According to embodiments of the present invention, the preferred anionic stabilizers sulfur dioxide. Other anionic stabilizers may be a very strong acid including without limitation perchloric acid, hydrochloric acid, hydrobromic acid, toluenesulfonic acid, fluorosulfonic acid, phosphoric acid, ortho, meta, or para-phosphoric acid, trichloroacetic acid, and sulfuric acid.

According to embodiments of the present invention, the compositions may optionally contain thickening agents. Suitable thickening agents include polycyanoacrylate, partial polymer of cyanoacrylate, polycaprolactone, copolymers of alkylacrylate and vinyl acetate, polyalkyl methacrylates, polyalkyl acrylates, lactic-glycolic acid copolymers, lactic acid-caprolactone copolymers, polyorthoesters, copolymers of alkyl methacrylates and butadiene, polyoxalates, and triblock copolymers of polyoxypropylene flanked by two hydrophilic chains of polyoxyethylene. According to certain embodiments of the present invention, a plasticizer may be included in the inventive cyanoacrylate compositions. The plasticizing agent preferably does not contain any moisture and should not adversely affect the stability of the cyanoacrylate compositions. Examples of suitable plasticizers include, but are not limited to, tributyl citrate (TBC), acetyl tributyl citrate (ATBC), dimethyl sebacate, diethylsebacate, triethyl phosphate, tri(2-ethyl-hexyl)phosphate, tri(p-cresyl) phosphate, diisodecyl adipate (DIDA), glyceryl triacetate, glyceryl tributyrate, dioctyl adipate (DICA), isopropyl myristate, butyl sterate, lauric acid, trioctyl trimellitate, dioctyl glutatrate (DICG) and mixtures thereof. Tributyl citrate, diisodecyl adipate and acetyl tributyl citrate are preferred plasticizers used in an amount of 0 to 30%, preferably 1% to 20%, and more preferably 2% to 10%.

The compositions of this invention may further contain small amounts of colorants such as dyes, pigments, and pigment dyes. Suitable dyes include derivatives of anthracene and other complex structures. These dyes include without limitation, 1-hydroxy-4-[4methylphenylamino]-9,10 anthracenedione (D&C violet No. 2); 9-(o-carboxyphenyl)-6-hydroxy2,4,5,7-tetraiodo-3H-xanthen-3-one-, disodium salt, monohydrate (FD&C Red No. 3); disodium salt of 6-hydroxy-5-[(4-sulfophenyl)axo]-2-naphthalene-sulfonic acid (FD&C Yellow No. 6); 2(1,3dihydro-3-oxo-5-sulfo-2H-indole-2-ylidine)-2,3-dihydro-3-oxo-1H-ind-ole-5 sulfonic acid disodium salt (FD&C Blue No. 2); and 1,4-bis(4-methylanilino)anthracene-9,10-dione (D&C Green No. 6).

In order to be used as catheter securement device, the liquid adhesive composition disclosed in the present patent should be sterile. Sterilization of the liquid adhesive composition disclosed herein can be accomplished by any method, including, but not limited to chemical, physical and irradiation techniques. Examples of chemical methods include, but are not limited to, exposure to ethylene oxide and hydrogen peroxide. Examples of physical methods include, but are not limited to, sterilization by heat (dry or moist) or retort canning. Examples of irradiation methods include, but are not limited to, gamma irradiation, electron beam irradiation, X-ray irradiation and microwave irradiation. Preferred methods of sterilization are irradiation methods, including but not limited to, electron beam (E-beam), gamma irradiation and X-Ray. The container and/or packaging for the inventive adhesive compositions may also be sterilized with heat or ethylene oxide prior to the final irradiation sterilization.

When sterilizing the compositions using E-beam irradiation, the dose applied which is sufficient enough to sterilize the adhesive compositions, typically, ranges from about 5 kGy to 50 kGy, and more preferably from about 5 kGy to 25 kGy. E-beam irradiation is preferably conducted at ambient atmosphere conditions and the exposure time to the irradiation is preferably within 60 seconds. Any standard power source is suitable, including a linear accelerator, which produces irradiation measured in kilo watts (KW). The larger the beam power, the more product volume can be processed. The inventive cyanoacrylate adhesive compositions are irradiated at a beam power ranging from about 2 KW to about 30 KW, preferably about 5 KW to about 20 KW, and more preferably about 10 KW to about 20 KW. E-beam irradiation typically involves the use of high-energy electrons. The beam energy ranges from 1 million to 10 million electron volts (MeV), preferably 3 MeV to 10 MeV, and more preferably 5 MeV to 10 MeV.

The dose of Gamma irradiation to the liquid adhesive composition disclosed in the present patent is in the range of about 5 kGy to about 40 kGy, preferably in the range of about 5 kGy to about 30 kGy, more preferably about 5 kGy to about 25 kGy, and most preferably about 5 kGy to about 20 kGy. The dose of X-ray irradiation to cyanoacrylate compositions with naphthoquinone 2,3-oxide is in the range of about 5 kGy to about 40 kGy, preferably in the range of about 5 kGy to about 30 kGy, more preferably about 5 kGy to about 25 kGy, and most preferably about 5 kGy to about 20 kGy. High energy electrons are used in the instant method of x-ray sterilization of liquid adhesive compositions. X-rays are generated as high-frequency and short-wavelength electromagnetic photons. Conventional x-ray technology is suitable in the instant invention. The X-ray energy used in the present invention ranges from 1 million to 10 million electron volts (MeV), preferably 3 MeV to 10 MeV, and more preferably 3 to 7.5 MeV. The dose of gamma irradiation desirably ranges from about 5 to about 25 kGy, preferably in the range of about 5 to about 20 kGy, and more preferably in the range of about 5 to about 15 kGy. Standard Cobalt Co-60 may be used as the gamma ray source in sterilizing the compositions and packages of the present invention.

After irradiation sterilization, the viscosity of the cyanoacrylate composition may change, including an increase or decrease. The change in viscosity of the cyanoacrylate adhesive compositions, after the sterilization, may vary, for example, depending on the original viscosity and the presence of additives such as a polymerization accelerator, a medicament, or stabilizers. The increase in viscosity of the composition after is within about 1% to about 200%, preferably within about 1% to about 80%, and more preferably within about 1% to about 60% of the initial viscosity of the composition, before sterilization. In some embodiments, the viscosity of the composition after sterilization is within about 5% to about 300% of the initial viscosity of the composition, before sterilization. The viscosity may change about 5% to about 10%, about 5% to about 15%, about 5% to about 20%, about 7% to about 10%, about 7% to about 15%, about 8% to about 12%, about 8% to about 15%, about 8% to about 20%, about 10% to about 100%, about 10% to about 80%, about 10% to about 60%, about 10% to about 40%, about 10% to about 30%, about 10% to about 20%, about 20% to about 100%, about 20% to about 60%, about 20% to about 50%, about 20% to about 40%, about 20% to about 30%, about 30% to about 300%, about 30% to about 200%, about 30% to about 150%, about 30% to about 100%, about 30% to about 50%, about 40% to about 300%, about 40% to about 200%, about 40% to about 150%, about 40% to about 100%, about 40% to about 80%, about 40% to about 80%, about 50% to about 300%, about 50% to about 200%, about 50% to about 150%, about 50% to about 100%, about 50% to about 90%, about 50% to about 80%, about 60% to about 200%, about 60% to about 100%, about 70% to about 200%, about 70% to about 100%, about 80% to about 100% of the initial viscosity.

An adhesive typically should have a shelf-life of at least one year to be used for medical purposes, however, an increased shelf-life beyond this provides increased economic advantages to both the manufacturer and the consumer. As used herein, shelf-life refers to the amount of time the composition therein can be held at ambient conditions (approximately room temperature) or less, without degradation of the composition occurring to the extent that the composition cannot be used in the manner and for the purpose for which they were intended. Thus, while some degradation to the composition can occur, it must not be to such an extent that the composition is no longer useable. As used herein, an “extended shelf-life” refers to a shelf-life of at least 12 months, preferably at least 18 months, and more preferably at least 24 months.

In order to evaluate the stability of the liquid adhesive composition disclosed in the present patent after sterilization, an accelerated aging test was performed. As used herein “stability” refers to the resultant composition maintaining a commercially acceptable form for the prescribed amount of time. That is, the composition does not prematurely polymerize or otherwise change form or degrade to the point that the composition is not useful for its intended purpose. Thus, while some polymerization or thickening of the composition may occur, such as can be measured by changes in viscosity of the composition, such change is not so extensive as to destroy or significantly impair the usefulness of the composition. The accelerated aging test was performed in the oven at 80° C. for a period of 13 days. Based on calculations, 13 days accelerated aging at 80° C. is equal to 2 years of shelf life, and 1 day of accelerated aging at 80° C. is equal to 56 days.

The stability of the liquid adhesive composition disclosed in the present patent after sterilization can also be evaluated by the real time assessment. The real time aging study was conducted at room temperature by keeping the cyanoacrylate compositions in the suitable package after irradiation sterilization in a designated environment where the temperature and humidity are monitored by a chart recorder. The temperature was controlled at 22° C.±5° C. and the humidity cannot exceed 80%. Viscosity, curing speed, and purity of the cyanoacrylate adhesive compositions in the suitable package after irradiation sterilization were evaluated at day 0, month 12, and 24 or other intervals between day 0 and month 24 to assess the performance and stability of the adhesive compositions. The viscosity was used to evaluate the stability of the adhesive compositions. Compared to the cyanoacrylate adhesive compositions disclosed herein after terminal sterilization at day 0, the viscosity of the cyanoacrylate compositions slightly increases as the real time/shelf life study reaches month 24, indicating a shelf life of at least 24 months.

The present invention further provides a kit for applying the cyanoacrylate adhesive composition, including an applicator containing therein an effective amount of the cyanoacrylate composition. The applicators are designed to be safe and easy to use, with a tapered tip and the ability to control the flow rate of an adhesive or sealant material for the purpose of precise control. The applicator is also compatible with radiation sterilization techniques. In particular, the containers for the adhesive and sealant material of the applicators are made of materials with high moisture and air barrier properties such as cyclic olefin copolymers or acrylonitrile copolymers so that the adhesive and sealant material can be sterilized by radiation and thereafter provide long-term shelf stability.

In a non-limiting embodiment the present invention includes: a body, a container for containing an adhesive material, and a rigid tapered tip. Adhesives may be pre-packaged in the applicator in the container, for example, sealed within the container by a frangible foil or a membrane, which may be hermetically sealed. The container for adhesives can be fabricated from a multi-layer sheet material, and the inner layer of the container, which contacts the adhesive, can be fabricated from a cyclic olefin copolymer. The container thereby constructed is compatible with radiation sterilization, such as electron beam, gamma, or x-ray sterilization, so that adhesives inside the applicator can be sterilized via radiation without diminished shelf stability (e.g., without prematurely polymerizing). The long-term shelf life stability of adhesive packaged in the applicators may be provided after radiation sterilization.

The container comprises a plurality of walls that define a chamber that is preferably open at the distal end, which may be closed off by at least one seal, for example, a frangible seal. The frangible seal is heat sealed to the container for storing the adhesive. Suitable materials for the frangible seal may include, but are not limited to, aluminum foil, plastic membrane, laminated aluminum foil, plastic wrap, waxed paper, oiled paper, or the like. Laminated aluminum foil may be composed of at least two layers of different materials which include, but are not limited to, aluminum, acrylonitrile copolymer, low density polyethylene, low density polypropylene, polyethylene teraphthalate, cyclic olefin copolymer, and the like.

Suitable materials for the container should have a desired barrier property for moisture and air so that the premature polymerization of the adhesive can be prevented or inhibited. The exceptional barrier properties offered by preferred materials of this invention make them ideal materials for use in construction of the packaging bodies in accordance with the present invention. Preferred materials of this invention offer a high barrier to oxygen at all levels of relative humidity. This ensures that a consistently high barrier to oxygen is maintained, regardless of the humidity of the surrounding environment. In addition, the water vapor barrier properties of the preferred materials of this invention make them desirable materials for packaging and sterilizing cyanoacrylate-based adhesive materials in accordance with the present invention. Suitable materials for the container include, but are not limited to, high density polyethylene (HDPE), polypropylene, polyvinylchloride, acrylonitrile copolymer, polycarbonate, polytetrafluoroethylene (PFTE), cyclicolefin copolymer, and the like.

Suitable materials for the container and the inner layer of the frangible seal include unsaturated cyclic monomers and one or more unsaturated linear monomer. Unsaturated linear monomers include without limitation alkenes having 1 to 20, preferably from 1 to 12 carbon atoms, most preferably from 1 to 6 carbon atoms, such as for example alpha-olefins, for example ethylene, propylene, and butylene. Unsaturated cyclic monomers include without limitation, cyclopentadiene and derivatives thereof such as for example dicyclopentadiene and 2,3-dihydrocyclopentadiene; 5,5-dimethyl-2-norbomene, 5-butyl-2-norbomene, 5-ethylidene-2-norbornene, norbornene and derivatives thereof, 2-norbomene, 5-methyl-2-norbomene, 5-methoxycarbonyl-2-norbornene, 5-cyano-2-norbornene, 5-methyl-5-methoxycarbonyl-2-norbornene, and 5-phenyl-2-norbornene, and combinations of two or more thereof. Other unsaturated linear monomers may be chosen from 1-butene, 4-methyl-1-pentene, 1-hexene, 1-octene, 1-decene, 1-dodecene, 1-tetradecene, 1-hexadecene, 1-octadecene and 1-eicocene, cydopentene, cydohexane, 3-methylcyclohexene, cyclooctene, 1,4-hexadiene, 4-methyl-1,4-hexadiene, 5-methyl-1,4-hexadiene, 1,7-octadiene, dicyclopentadiene, 5-ethylidene-2-norbornene, 5-vinyl-2-norbornene, tetracyclododecene, 2-methyltetracyclododecene, and 2-ethyltetracyclododecene; or combinations of two or more thereof. Preferably the unsaturated linear monomer is 1-hexene, butylene, propylene, and ethylene. Preferably the copolymer is cyclopentadiene-ethylene copolymer, cyclopentadiene-butylene copolymer, cyclopentadiene-hexene copolymer, cyclopentadiene-propylene copolymer, cyclopentadiene-octene copolymer, dicyclopentadiene-ethylene copolymer, dicyclopentadiene-butylene copolymer, dicyclopentadiene-hexene copolymer, dicyclopentadiene-propylene copolymer, dicyclopentadiene-octene copolymer, norbornene-ethylene copolymer, norbomene-propylene copolymer, norbornene-butylene copolymer, norborene-hexene copolymer, 5-cyano-2-norbornene-ethylene copolymer, 5-cyano-2-norbornene-propylene copolymer, 5-cyano-2-norbornene-butylene copolymer, 5-phenyl-2-norbornene-ethylene copolymer, 5-phenyl-2-norbomene-propylene copolymer, 5-phenyl-2-norbornene-butylene copolymer, 5-methyl-5-methoxycarbonyl-2-norbornene-ethylene copolymer, 5-methyl-5-methoxycarbonyl-2-norbornene-propylene copolymer, 5-methyl-5-methoxycarbonyl-2-norbornene-butylene copolymer, 5-ethylidene-2-norbornene-ethylene copolymer, 5-ethylidene-2-norbornene-propylene copolymer, and 5-ethylidene-2-norbomene-butylene copolymer, acrylonitrile copolymers produced by polymerizing a major proportion of a monounsaturated nitrile and a minor proportion of another monovinyl monomer or indene copolymerizable nitrile polymers produced by polymerizing a major portion of a monounsaturated nitrile and a minor portion of another monovinyl monomer or indene copolymerizable therewith in the presence of a diene rubber, polyacrylates, polymethacrylate, polyalkyl methacrylates, polyethers, polysiloxanes, polysulfones, polyphenylene sulfide, polyether ether ketones, thermoplastic polyimides, polybenzimidazoles, polyquinoxalines, polyoxazolines, styrene-acrylonitrile copolymer and acrylonitrile-butadiene-styrene copolymer, vinyl acetate containing polymers, maleic anhydride containing polymers, butadiene and/or isoprene based elastomers, acrylonitrile, and methacrylonitrile.

The applicator body may be fabricated from any suitable materials. A cutting portion inside the applicator body is designed to be sharp and strong so as to readily break the frangible seal for dispensing adhesive inside the container. Conversely, the grip section should be flexible and soft enough to make squeezing readily possible. Therefore, the material of the applicator body is specifically designed for the applicator body. Suitable materials for the applicator body include but are not limited to polyethylene (PE), polypropylene, polyvinylchloride, polycarbonate, polytetrafluoroethylene (PFTE), polystyrene (PS), and polymethylpentene with a certain percentage of thermoplastic elastomers (TPE). TPE may be present in the materials used for constructing the applicator body in the amount of 2% to 70%, preferably 3% to 60%, and more preferably 5% to 50%. Without including TPE, the applicator body is too hard to be squeezed to dispense and control the flow of the adhesive. If too much TPE is present in the applicator body, the cutting portion becomes too soft to puncture through the frangible seal for dispensing adhesive inside the container.

In preferred embodiments of the present invention, the composition is sterilized to provide a Sterility Assurance Level (SAL) of at least 10⁻³. This means that the probability of a single unit being non-sterile after sterilization is 1 in 1000. In more preferred embodiments, the sterility assurance level may be at least 10⁻⁴, 10⁻⁵, or 10⁻⁶. After sterilizing the inventive cyanoacrylate adhesive compositions, their sterility levels were analyzed by Bacteriostasis and Fungistasis tests. After testing with challenging microorganisms such as Bacillus subtilis, Candida albicans, and Aspergillus niger, no growth of the microorganisms was observed, indicating the sterility of the inventive compositions.

Additional objects, advantages, and novel features of this invention will become apparent to those skilled in the art upon examination of the following examples of the invention. The examples are included to more clearly demonstrate the overall nature of the invention and, thus, are illustrative and not restrictive of the invention.

### EXAMPLES

The following non-limiting examples are intended to further illustrate, but not to limit, the invention.

### Example 1

Securement strength of Tegaderm™ 9525HP vs. the liquid adhesive composition disclosed in the present patent for Securing BD Nexiva Catheters after 6 hours. Porcine skin was prepared and cleaned with isopropyl alcohol. Measure and mark a spot to insert the catheter in the center of the skin. Inject the IV Catheter needle into the skin as to leave the IV Catheter resting flush against the skin and the catheter tube injected into the skin. To secure the BD Nexiva Catheter with Tegaderm™ 9525HP, apply Tegaderm™ 9525HP per IFU. To secure the BD Nexiva Catheter with the liquid adhesive composition disclosed in the present patent, apply the adhesive under the catheter hub, tubing, and insertion site. Once the liquid adhesive composition disclosed in the present patent has been applied, hold the BD Nexiva Catheter in place for four minutes to ensure bonding between the catheter and the skin. After the BD Nexiva Catheter has been secured let the sample rest for 6 hours at room temperature prior to testing the securement strength on the Tensiometer. This test was repeated for a total of ten samples. The average pounds-force required to remove the BD Nexiva Catheter from the pig skin when Secured with Tegaderm™ 9525HP was 1.68 lbf compared to the liquid adhesive composition disclosed in the present patent which was 3.72 lbf.

### Example 2

Securement strength of Opsite Alone vs. the liquid adhesive composition disclosed in the present patent for Securing BD Nexiva Catheters after 30 minutes. To begin, prepare porcine skin by cutting and cleaning the skin with isopropyl alcohol. Measure and mark a spot to insert the catheter in the center of the skin. Inject the IV Catheter needle into the skin as to leave the IV Catheter resting flush against the skin and the catheter tube injected into the skin. To secure the BD Nexiva Catheter with Opsite alone, apply Opsite per IFU. To secure the BD Nexiva Catheter with the liquid adhesive composition disclosed in the present patent, apply the adhesive under the catheter hub, tubing, and insertion site. Once the liquid adhesive composition disclosed in the present patent has been applied, hold the BD Nexiva Catheter in place for four minutes to ensure bonding between the catheter and the skin. After the BD Nexiva Catheter has been secured let the sample rest for 30 minutes at room temperature prior to testing the securement strength on the Tensiometer. This test was repeated for a total of ten samples. The average pounds-force required to remove the BD Nexiva Catheter from the pig skin when secured with Opsite and the liquid adhesive composition disclosed in the present patent was 0.31, and 3.0 lbf, respectively.

### Example 3

Securement strength of Durapore™ vs. the liquid adhesive composition disclosed in the present patent for Securing BD Autoguard Catheters after 3 minutes. To begin, prepare porcine skin by cutting and cleaning the skin with isopropyl alcohol. Measure and mark a spot to insert the catheter in the center of the skin. Inject the IV Catheter needle into the skin as to leave the IV Catheter resting flush against the skin and the catheter tube injected into the skin. To secure the BD Autoguard Catheter with Durapore™ alone, apply Durapore™ per IFU. To secure the BD Autoguard Catheter with the liquid adhesive composition disclosed in the present patent, apply the adhesive under the catheter hub, tubing, and insertion site. Once the liquid adhesive composition disclosed in the present patent has been applied, hold the BD AutoGuard Catheter in place for four minutes to ensure bonding between the catheter and the skin. After the BD Autoguard Catheter has been secured let the sample rest for 3 minutes at room temperature prior to testing the securement strength on the Tensiometer. This test was repeated for a total of ten samples for each article. The average pounds-force required to remove the BD AutoGuard Catheter from the pig skin when secured with Durapore™ and the liquid adhesive composition disclosed in the present patent was 0.79, and 1.01 lbf, respectively.

### Example 4

Securement strength of the liquid adhesive composition disclosed in the present patent+Tegaderm™ 9525HP for securing BD Nexiva Catheters after 6 hours. To begin, prepare porcine skin by cutting and cleaning the skin with isopropyl alcohol. Measure and mark a spot to insert the catheter in the center of the skin. Inject the IV Catheter needle into the skin as to leave the IV Catheter resting flush against the skin and the catheter tube injected into the skin. To secure the BD Nexiva Catheter, apply the liquid adhesive composition disclosed in the present patent under the catheter hub, tubing, and insertion site. Once the liquid adhesive composition disclosed in the present patent has been applied, apply Tegaderm™ 9525HP, per IFU. After both the liquid adhesive composition disclosed in the present patent and Tegaderm™ 9525HP are applied to the sample let the sample rest for 6 hours at room temperature prior to testing the securement strength on the Tensiometer. This test was repeated for a total of ten samples and the average pounds-force required to remove the BD Nexiva Catheter from the pig skin when secured with the liquid adhesive composition disclosed in the present patent+Tegaderm™ 9525HP was 3.60 lbf.

### Example 5

Securement strength of the liquid adhesive composition disclosed in the present patent+Opsite for securing BD AutoGuard Catheters after 30 Minutes. To begin, prepare porcine skin by cutting and cleaning the skin with isopropyl alcohol. Measure and mark a spot to insert the catheter in the center of the skin. Inject the IV Catheter needle into the skin as to leave the IV Catheter resting flush against the skin and the catheter tube injected into the skin. To secure the BD Autoguard Catheter, apply the liquid adhesive composition disclosed in the present patent under the catheter hub, tubing and insertion site. Once the the liquid adhesive composition disclosed in the present patent has been applied, apply Opsite, per IFU. After both the liquid adhesive composition disclosed in the present patent and Opsite are applied to the sample let the sample rest for 30 minutes at room testing prior to testing the securement strength on the Tensiometer. This test was repeated for a total of ten samples and the average pounds-force required to remove the BD Autoguard Catheter from the pig skin when secured with the liquid adhesive composition disclosed in the present patent+Opsite was 3.47 lbf.

### Example 6

Securement strength of the liquid adhesive composition disclosed in the present patent for securing BD Nexiva Catheters after 1,3, & 7 Days. To begin, prepare porcine skin by cutting and cleaning the skin with isopropyl alcohol. Measure and mark a spot to insert the catheter in the center of the skin. Inject the IV Catheter needle into the skin as to leave the IV Catheter resting flush against the skin and the catheter tube injected into the skin. To secure the BD NexivaCatheter, apply the liquid adhesive composition disclosed in the present patent under the catheter hub, tubing and insertion site. Once the liquid adhesive composition disclosed in the present patent has been applied, hold the BD Nexiva Catheter in place for four minutes to ensure bonding between the catheter and the skin. After the BD Nexiva Catheter has been secured let the sample rest for 1 day, 3 days or 7 days in the refrigerator prior to testing the securement strength on the Tensiometer. This test was repeated for a total of ten samples each time point (1 day, 3 days, & 7 days). The average pounds-force required to remove the BD Nexiva Catheter from the pig skin when secured with the liquid adhesive composition disclosed in the present patent alone at 1 day, 3 Days, and 7 Days was 3.06 lbf, 3.02 lbf, and 3.72 lbf, respectively.

### Example 7

This study is to evaluate the peel strength of the liquid adhesive composition disclosed in the present patent+Tegaderm™ 9525HP with additional drops of the liquid adhesive composition disclosed in the present patent applied under the Tegaderm™ 9525HP window while securing BD Nexiva Catheters to porcine skin. To begin, prepare porcine skin by cutting and cleaning the skin with isopropyl alcohol. Measure and mark a spot to insert the catheter in the center of the skin. Inject the IV Catheter needle into the skin as to leave the IV Catheter resting flush against the skin and the catheter tube injected into the skin. To secure the BD Nexiva Catheter, apply the liquid adhesive composition disclosed in the present patent under the catheter hub, tubing and insertion site. Apply additional drops and spread to cover the area where the window section of the Tegaderm™ 9525HP dressing will be applied. Once the liquid adhesive composition disclosed in the present patent has been applied, immediately apply Tegaderm™ 9525HP, per IFU. After both the liquid adhesive composition disclosed in the present patent and Tegaderm™ 9525HP are applied to the sample let the sample rest for 30 minutes at room temperature prior to testing the peel strength on the Tensiometer. This test was repeated for a total of ten samples and the average peel strength after 30 minutes for Tegaderm™ 9525HP secured by additional liquid adhesive composition disclosed in the present patent drops spread under the window area with Tegaderm™ 9525HP applied immediately was NLT 12.2 lbf.

### Example 8

This study is to evaluate the securement strength of BD Nexiva Catheters secured to porcine skin by the liquid adhesive composition disclosed in the present patent plus Tegaderm™9525HP with additional drops of the liquid adhesive composition disclosed in the present patent applied under the entire area of the Tegaderm™9525HP dressing. To begin, prepare porcine skin by cutting and cleaning the skin with isopropyl alcohol. Measure and mark a spot to insert the catheter in the center of the skin. Inject the IV Catheter needle into the skin as to leave the IV Catheter resting flush against the skin and the catheter tube injected into the skin. To secure the BD Nexiva Catheter, apply the liquid adhesive composition disclosed in the present patent under the catheter hub, tubing and insertion site. Apply additional drops and spread to cover the entire area where the Tegaderm™ 9525HP dressing will be applied. Once the liquid adhesive composition disclosed in the present patent has been applied, immediately apply Tegaderm™ 9525HP, per IFU. After both the liquid adhesive composition disclosed in the present patent and Tegaderm™ 9525HP are applied to the sample let the sample rest for 30 minutes at room temperature prior to testing the securement strength on the Tensiometer. This test was repeated fora total of ten samples and the average securement strength after 30 minutes for Nexiva catheter secured by additional liquid adhesive composition disclosed in the present patent spread under the entire area of the Tegaderm™ 9525HP dressing with Tegaderm™ 9525HP applied immediately was 11.8 lbf.

### Example 9

This study is to evaluate the peel strength of the liquid adhesive composition disclosed in the present patent plus Tegaderm™ 9525HP with additional drops of the liquid adhesive composition disclosed in the present patent applied under the entire area of the Tegaderm™9525HP dressing while securing BD Nexiva Catheters to porcine skin. To begin, prepare porcine skin by cutting and cleaning the skin with isopropyl alcohol. Measure and mark a spot to insert the catheter in the center of the skin. Inject the IV Catheter needle into the skin as to leave the IV Catheter resting flush against the skin and the catheter tube injected into the skin. To secure the BD Nexiva Catheter, apply the liquid adhesive composition disclosed in the present patent under the catheter hub, tubing and insertion site. Apply additional drops and spread to cover the entire area where the Tegaderm™ 9525HP dressing will be applied. Once the liquid adhesive composition disclosed in the present patent has been applied, immediately apply Tegaderm™ 9525HP, per IFU. After both the liquid adhesive composition disclosed in the present patent and Tegaderm™ 9525HP are applied to the sample let the sample rest for 30 minutes at room temperature prior to testing the peel strength on the Tensiometer. This test was repeated for a total of ten samples and the average peel strength after 30 minutes for Tegaderm™9525HP secured by additional liquid adhesive composition disclosed in the present patent spread under the entire area of the Tegaderm™ 9525HP dressing with Tegaderm™ 9525HP applied immediately was NLT 14.9 lbf.

### Example 10

The sealant integrity of the liquid adhesive composition disclosed in the present patent for securing BD Autoguard Catheters onto porcine skin for 1 day, 4 days and 7 days was investigated. Prepare porcine skin by cutting and cleaning the skin with isopropyl alcohol. Measure and mark a spot to insert the catheter in the center of the skin. Inject the IV Catheter needle into the skin as to leave the IV Catheter resting flush against the skin and the catheter tube injected into the skin. Measure and mark a rectangular area surrounding the catheter as a guide to spread the adhesive. Apply the liquid adhesive composition disclosed in the present patent under the catheter hub and tubing as well as spreading to cover the complete rectangular area surrounding the catheter. Once the liquid adhesive composition disclosed in the present patent has been applied, hold the BD Autoguard Catheter in place for four minutes to ensure bonding between the catheter and the skin. After four minutes has past let the sample dry an additional twenty-six minutes before adding the liquid dye over the insertion site. The samples were left to rest in the refrigerator until the correct time point. This test was repeated for a total of ten samples for each time point (1 day, 3 days, & 7 days). The water based liquid dye did not penetrate the film layer at the junction of the catheter and the skin at any of the three time points. Therefore, the liquid adhesive composition disclosed in the present patent provides an effective barrier against aqueous solutions for up to 7 days.

### Example 11

The catheter securement capability of the liquid adhesive composition disclosed in the present patent was also evaluated in the canine model on securing three different types of cannulas in peripheral and central veins. A total of six dogs were enrolled in this study and divided into 3 groups with two animals per group. Either Insyte Autoguard IV Catheters, Power PICC Dual-Lumen Catheters, or Arrowgard Coated Polyurethane CVCs were implanted into the animals representing the three different groups. Each animal had two of the same type catheters implanted in either jugular veins (Power PICC or Arrowgard) or cephalic veins (Autoguard) and the liquid adhesive composition disclosed in the present patent was applied to each insertion site to secure the catheter. After achieving cannula securement, animals were recovered from anesthesia and a weight stress (manual tug test) was applied hourly on the catheter for a total of six hours to assess the performance of the test article. The catheters were adhered to the animal with the liquid adhesive composition disclosed in the present patent alone for the first three hours (no additional adhesive bandaging used). After the hour three testing and before hour four testing, a Tegaderm™ 1683 bandage was placed over top of the already secured catheter. The tug test was performed with Tegaderm™ 1683 present for the last three data points. The liquid adhesive composition disclosed in the present patent successfully secured all three types of catheters tested in peripheral and central veins for a minimal of three hours without dressing product and in combination with Tegaderm™ 1683 for six hours.

### Example 12

The ability of the liquid adhesive composition disclosed in the present patent to immobilize bacteria, preventing the spread of the most motile bacteria including Escherichia coli, Proteus mirabilis, and Serratia marcescens, was demonstrated in this study. The cultures of Escherichia coli, Proteus mirabilis, and Serratia marcescens were grown with tryptic soy broth (TSB) at 32.5° C.±2.5° C. or 20° C. 2.5° C. for 18-24 hours prior to use and then diluted with 0.85% sterile saline. The concentration of the diluted cultures was verified by plating 2×100 μL of dilutions onto tryptic soy agar (TSA). In duplicate, the TSA plates were challenged with ˜100-500 cfu (10 μL) of the challenge organism and the liquid adhesive composition disclosed in the present patent was immediately applied over the challenge organism. The inoculum and glue were allowed to dry and were then incubated, at 30-35° C. (Escherichia coli and Proteus mirabilis) and 20-25° C. (Serratia marcescens). A positive control and a negative control were also tested and all plates were monitored for up to 48 hrs. The mm of horizontal growth/spread were measured for the test article and the positive control. The positive control demonstrated the movement of the challenge organisms with results of 1.5 mm. 6.5 mm, and 0.5 mm of growth spread for Escherichia coli, Proteus mirabilis, and Serratia marcescens, respectively. When applying the liquid adhesive composition disclosed in the present patent, the results decreased to 0 mm of horizontal growth or spreading of challenge bacteria proving that the liquid adhesive composition disclosed in the present patent successfully immobilized all three test organisms including the highly motile Proteus mirabilis. In immobilizing bacteria, the liquid adhesive composition disclosed in the present patent has the potential to mitigate the risk of infection at the insertion site, near open wounds or damaged skin providing protection to the overall vascular access site.

### Example 13

The stability of a liquid adhesive composition based upon 2-octyl cyanoacrylate post Gamma and EO sterilization was evaluated by the real time assessment. The real time aging study was conducted at room temperature where the temperature and humidity are monitored by a chart recorder. The temperature was controlled at 22° C.±5° C. and the humidity cannot exceed 80%. Viscosity and set time of the cyanoacrylate adhesive composition were evaluated at day 0, month 12, and 24 or other intervals between day 0 and month 24 to assess the performance and stability of the adhesive compositions. The average viscosity and set time for the liquid adhesive composition was 6.0 cps, and 18 seconds, respectively, at day 0, which were slightly increased to 48.4 cps and 44 seconds, respectively, at real time shelf month 24. Both the viscosity and set time throughout the 24 months of real tie shelf life have remained within the specifications (200 cps, and 90 seconds, respectively).

### Example 14

The stability of a liquid adhesive composition based upon a mixture of 2-octyl cyanoacrylate and n-butyl cyanoacrylate post Gamma and EO sterilization was evaluated by the real time assessment. The real time aging study was conducted at room temperature where the temperature and humidity are monitored by a chart recorder. The temperature was controlled at 22° C. 5° C. and the humidity cannot exceed 80%. Viscosity and set time of the cyanoacrylate adhesive composition were evaluated at day 0, month 12, and 24 or other intervals between day 0 and month 24 to assess the performance and stability of the adhesive compositions. The average viscosity and set time for the liquid adhesive composition was 5.33 cps, and 17.5 seconds, respectively, at day 0, which were slightly increased to 9.35 cps and 35 seconds, respectively, at real time shelf month 24. Both the viscosity and set time throughout the 24 months of real tie shelf life have remained within the specifications (200 cps, and 90 seconds, respectively).

What is claimed is:

1. A method for securing a vascular access device which has a tube that has been inserted into a vascular system of a patient at a vascular access point, the method comprising:
applying a first amount of a radiation-sterilized cyanoacrylate adhesive composition over the vascular access point;
allowing the first amount of the radiation-sterilized cyanoacrylate adhesive composition to cure to create a first cured radiation-sterilized cyanoacrylate adhesive composition, wherein the first cured radiation-sterilized cyanoacrylate adhesive composition is transparent and provides a water-resistant seal barrier, a hemostatic effect on the vascular access point, an antimicrobial function to immobilize and eliminate bacteria at and around vascular access point, and secures the insertion tube to the vascular access site;
applying a second amount of the radiation-sterilized cyanoacrylate adhesive composition under a portion of the vascular access device at a site remote from the vascular access point; and
allowing the second amount of the radiation-sterilized cyanoacrylate adhesive composition to cure and secure the vascular access device to the patient with a secured strength;
wherein the secured strength of the secured vascular access device to the patient is greater than 2 pounds of force (lbf).

2. The method according to claim 1, wherein the water-resistant seal barrier lasts for more than 3 days.

3. The method according to claim 1, wherein the vascular access device is a catheter and the first and second cured radiation-sterilized cyanoacrylate adhesive compositions further inhibits complications associated with catheter insertion selected from the group consisting of catheter dislodgement, catheter infiltration, catheter migration, catheterocclusion, catheter-related phlebitis, and catheter-related infections.

4. The method according to claim 1, wherein the first cured radiation-sterilized cyanoacrylate adhesive composition further prevents bleeding at the vascular access point.

5. The method according to claim 1, wherein the first and second cured radiation-sterilized cyanoacrylate adhesive compositions further immobilize bacteria at and around the vascular access point.

6. The method according to claim 1, wherein the first and second cured radiation-sterilized cyanoacrylate adhesive compositions are antimicrobials that provides at least a 6 log reduction of gram-positive bacteria, gram-negative bacteria, yeast, and fungi 3 minutes after curing.

7. The method according to claim 1, wherein the portion of the vascular access device under which second amount of the radiation-sterilized cyanoacrylate adhesive composition are wings or catheter hubs.

8. The method according to claim 1, wherein the secured strength of the secured vascular access device to the patient is greater than 3 lbf.

9. The method according to claim 1, wherein the secured strength of the secured vascular access device to the patient is greater than 5 lbf.

10. The method according to claim 1, wherein the vascular access device is selected from a group consisting of, but are not limited to: intravenous (IV) catheters; peripheral venous catheters (PVCs), central venous catheters (CVCs), peripherally inserted central catheters (PICCs), arterial catheters, urinary catheters, and dialysis catheters.

11. The method according to claim 1, wherein the radiation-sterilized cyanoacrylate adhesive composition comprises a stabilized and sterile polymerizable monomers or the mixture of stabilized and sterile polymerizable monomers.

12. The method according to claim 11, wherein the polymerizable monomers comprise a cyanoacrylate with straight chain or branched chain alkyl or alkoxyalkyl groups having 4 to 20 carbon atoms, including but not limited to, 2-octyl cyanoacrylate, dodecylcyanoacrylate, undecyl cyanoacrylate, decyl cyanoacrylate, butyl cyanoacrylate, nonyl cyanoacrylate, heptyl cyanoacrylate, hexyl cyanoacrylate, pentyl cyanoacrylate, propyl cyanoacrylate, ethyl cyanoacrylate, 2-ethylhexyl cyanoacrylate, methyl cyanoacrylate, 3-methoxybutyl cyanoacrylate, 2-butoxyethyl cyanoacrylate, 2-isopropoxyethyl cyanoacrylate, or 1-methoxy-2-propyl cyanoacrylate, or a combination thereof.

13. The method according to claim 11, wherein the radiation-sterilized cyanoacrylate adhesive composition is stabilized by free radical stabilizers and anionic stabilizers.

14. The method according to claim 11, wherein the radiation-sterilized cyanoacrylate adhesive composition is sterilized by irradiation methods and/or chemical sterilization methods.

15. The method according to claim 11, wherein the radiation-sterilized cyanoacrylate adhesive composition has a shelf life of at least 24 months post sterilization evaluated by real time shelf stability studies.

16. The method according to claim 1, wherein the vascular access device is selected from a group consisting of intravenous (IV) catheters; peripheral venous catheters (PVCs), central venous catheters (CVCs), peripherally inserted central catheters (PICCs), arterial catheters, urinary catheters, and dialysis catheters.

17. The method according to claim 1, wherein the radiation-sterilized cyanoacrylate adhesive provides securement of the vascular access device to the patient for up to 7 days.

18. The method according to claim 1, wherein the radiation-sterilized cyanoacrylate adhesive is packaged in an applicator.

19. The method according to claim 18, wherein the applicator is fabricated from a material that is substantially impermeable to moisture and air.

20. The method according to claim 1, wherein the radiation-sterilized cyanoacrylate adhesive compositions stop bleeding at the vascular access site, which can help reduce early dressing changes.

21. The method according to claim 1, wherein the radiation-sterilized cyanoacrylate adhesive compositions can be used alone and in combination with traditional securement devices or dressing products.

22. The method according to claim 1, wherein the radiation-sterilized cyanoacrylate adhesive compositions are compatible with conventional securement devices and dressing products, and the securement strength of said adhesive compositions plus conventional dressing products is stronger than the said adhesive compositions or conventional dressing products being used alone.

23. The method according to claim 1, wherein the secured strength for securing vascular access devices can further be significantly improved when the radiation-sterilized cyanoacrylate adhesive compositions are applied to vascular access device as well as to the conventional dressing products.

24. The method according to claim 23, wherein the secured strength for securing vascular access devices can be up to 15 lbf when the radiation-sterilized cyanoacrylate adhesive compositions are applied to vascular access device as well as to the conventional dressing products.

25. The method according to claim 1, wherein the radiation-sterilized cyanoacrylate adhesive compositions can seal vascular access sites and secure vascular access devices providing antibacterial property and stopping bleeding at or around vascular access sites, which usually requires three or more different conventional products to achieve.
